(19) United States
(12) Patent Application Publication
(10) Pub. No.: US 2002/0026225 A1
(43) Pub. Date: Feb. 28, 2002

(54) Diode laser irradiation and electrotherapy system for biological tissue stimulation

(76) Inventor: Kim Robin Segal, Dallas, TX (US)

Correspondence Address: HAYNES AND BOONE, LLP, 901 MAIN STREET, SUITE 3100, DALLAS, TX, 75202, US

(21) Appl. No.: 09/953,038

(22) Filed: Sep. 12, 2001

Related U.S. Application Data
(62) Division of application No. 09/003,665, filed on Jan. 7, 1998.
Application No. 09/003,665 is a continuation-in-part of application No. 08/621,950, filed on Mar. 25, 1996, now Pat. No. 5,755,752.
Application No. 08/621,950 is a continuation-in-part of application No. 07/873,385, filed on Apr. 24, 1992, now abandoned.

(51) Int. Cl. A61B 18/20

(52) U.S. Cl. 607/89

(57) ABSTRACT

An irradiation and electrotherapy system for treating biological tissue of a subject without exposing the tissue to damaging effects. The system includes a manipulable wand for contact with the tissue, a diode laser disposed in the wand for irradiating the tissue with coherent optical energy, a metal sheath for providing electrical stimulation to the tissue, and setting controls for operating the wand to achieve a rate of absorption and conversion to heat in the irradiated tissue in a range between a minimum rate sufficient to elevate the average temperature of the irradiated tissue to a level above the basal body temperature of the subject, and a maximum rate which is less than the rate at which the irradiated tissue is converted into a collagenous substance.

### CROSS REFERENCE TO RELATED APPLICATION

This is a continuation-in-part of currently pending U.S. patent application Ser. No. 08/621,950 filed on Mar. 24, 1996, which was a continuation-in-part of now abandoned U.S. Ser. No. 07/873,385 filed on Apr. 24, 1992, all of which are incorporated herein by reference.

### BACKGROUND OF THE INVENTION

The present invention relates generally to the treatment of living biological tissue by optical irradiation, and in particular to a system for stimulating soft, living tissue by diode laser irradiation and electrical stimulation.

Various non-surgical means have been employed in the therapeutic treatment of living tissue. Such techniques have included the application of ultrasonic energy, electrical stimulation, high frequency stimulation by diathermy, X-rays and microwave irradiation. While these techniques have shown some therapeutic benefit, their use has been somewhat limited because they generate excessive thermal energy which can damage tissue. Consequently, the energy levels associated with therapeutic treatments involving diathermy, X-ray, microwave and electrical stimulation have been limited to such low levels that little or no benefit has been obtained. Moreover, the dosage or exposure to microwaves and X-ray radiation must be carefully controlled to avoid causing health problems related to the radiation they generate. Ultrasonic energy is non-preferentially absorbed and affects all of the tissue surrounding the area to which it is directed.

Optical energy generated by lasers has been used for various medical and surgical purposes because laser light, as a result of its monochromatic and coherent nature, can be selectively absorbed by living tissue. The absorption of the optical energy from laser light depends upon certain characteristics of the wavelength of the light and properties of the irradiated tissue, including reflectivity, absorption coefficient, scattering coefficient, thermal conductivity, and thermal diffusion constant. The reflectivity, absorption coefficient, and scattering coefficient are dependent upon the wavelength of the optical radiation. The absorption coefficient is known to depend upon such factors as interband transition, free electron absorption, grid absorption (photon absorption), and impurity absorption, which are also dependent upon the wavelength of the optical radiation.

In living tissue, water is a predominant component and has, in the infrared portion of the electromagnetic spectrum, an absorption band determined by the vibration of water molecules. In the visible portion of the spectrum, there exists absorption due to the presence of hemoglobin. Further, the scattering coefficient in living tissue is a dominant factor.

Thus, for a given tissue type, the laser light may propagate through the tissue substantially unattenuated, or may be almost entirely absorbed. The extent to which the tissue is heated and ultimately destroyed depends on the extent to which it absorbs the optical energy. It is generally preferred that the laser light be essentially transmissive through tissues which are not to be affected, and absorbed by tissues which are to be affected. For example, when applying laser radiation to a region of tissue permeated with water or blood, it is desired that the optical energy not be absorbed by the water or blood, thereby permitting the laser energy to be directed specifically to the tissue to be treated. Another advantage of laser treatment is that the optical energy can be delivered to the treatment tissues in a precise, well defined location and at predetermined, limited energy levels.

Ruby and argon lasers are known to emit optical energy in the visible portion of the electromagnetic spectrum, and have been used successfully in the field of ophthalmology to reattach retinas to the underlying choroidea and to treat glaucoma by perforating anterior portions of the eye to relieve interoccular pressure. The ruby laser energy has a wavelength of 694 nanometers (nm) and is in the red portion of the visible spectrum. The argon laser emits energy at 488 nm and 515 nm and thus appears in the blue-green portion of the visible spectrum. The ruby and argon laser beams are minimally absorbed by water, but are intensely absorbed by blood chromogen hemoglobin. Thus, the ruby and argon laser energy is poorly absorbed by non-pigmented tissue such as the cornea, lens and vitreous humor of the eye, but is absorbed very well by the pigmented retina where it can then exert a thermal effect.

Another type of laser which has been adapted for surgical use is the carbon dioxide (CO₂) gas laser which emits an optical beam which is absorbed very well by water. The wavelength of the CO₂ laser is 10,600 nm and therefore lies in the invisible, far infrared region of the electromagnetic spectrum, and is absorbed independently of tissue color by all soft tissues having a high water content. Thus, the CO₂ laser makes an excellent surgical scalpel and vaporizer. Since it is completely absorbed, its depth of penetration is shallow and can be precisely controlled with respect to the surface of the tissue being treated. The CO₂ laser is thus well-suited for use in various surgical procedures in which it is necessary to vaporize or coagulate neutral tissue with minimal thermal damage to nearby tissues.

Another laser in widespread use is the neodymium doped yttrium-aluminum-garnet (Nd:YAG) laser. The Nd:YAG laser has a predominant mode of operation at a wavelength of 1064 nm in the near infrared region of the electromagnetic spectrum. The Nd:YAG optical emission is absorbed to a greater extent by blood than by water making it useful for coagulating large, bleeding vessels. The Nd:YAG laser has been transmitted through endoscopes for treatment of a variety of gastrointestinal bleeding lesions, such as esophageal varices, peptic ulcers, and arteriovenous anomalies.

The foregoing applications of laser energy are thus well-suited for use as a surgical scalpel and in situations where high energy thermal effects are desired, such as tissue vaporization, tissue cauterization, and coagulation.

Although the foregoing laser systems perform well, they commonly generate large quantities of heat and require a number of lenses and mirrors to properly direct the laser light and, accordingly, are relatively large, unwieldy, and expensive. These problems are somewhat alleviated insome systems by locating a source of laser light distal from a region of tissue to be treated and providing fiber optic cable for carrying light generated from the source to the tissue region, thereby obviating the need for a laser light source proximal to the tissue region. Such systems, however, are still relatively large and unwieldy and, furthermore, are much more expensive to manufacture than a system which does not utilize fiber optic cable. Moreover, the foregoing systems generate thermal effects which can damage living tissue, rather then provide therapeutic treatment to the tissue.

Therefore, what is needed is a system and method for economically stimulating soft, living tissue with laser energy without damaging the tissue from the thermal effects of the laser energy.

### SUMMARY OF THE INVENTION

The present invention, accordingly, provides a system and a method that retains all of the advantages of the foregoing systems while reducing the size and cost of the system. To this end, a system for treating biological tissue of a subject without exposing the tissue to damaging effects, the system includes a manipulable wand for contact with the tissue; a diode laser for emitting irradiation light disposed in the wand for irradiating the tissue with coherent optical energy; and setting controls coupled to the wand for operating the irradiation emitted from the laser diode to achieve a rate of absorption and conversion to heat in the irradiated tissue in a range between a minimum rate sufficient to elevate the average temperature of the irradiated tissue to a level above the basal body temperature of the subject, and a maximum rate which is less than the rate at which the irradiated tissue is converted into a collagenous substance. The coherent optical energy radiation can be combined with electrical stimulation using electrical current of less than 500 croamps to improve depth of penetration in the tissue.

In yet another aspect of the present invention, the amount of Indium with which the Gallium Arsenide in the diode is doped is appropriate to cause the wavelength of laser light generated by the diode to be in a range between 1064±20 nm and 2500±20 nm.

The system and method additionally enable the treatment time, the power generated by the laser, the electrical current levers, and the mode of operation (pulsed or continuous wattage (CW)) to be carefully controlled by an operator according to a desired treatment protocol.

An advantage achieved with the present invention is that it enables laser light and/or electrical stimulation to be safely and effectively applied to a region of living tissue for therapeutic purposes, for example, to reduce pain, reduce inflammation, and enhance the healing of tissue by stimulation of microcirculation, without exposing the tissue to damaging thermal and electrical effects.

Another advantage of the present invention is that it is less expensive to manufacture than systems utilizing fiber optic cables because the laser light is generated within the wand.

Yet, another advantage of the present invention is that it provides for high power dissipation levels up to about 1000 mw in both continuous wattage (CW) or pulsed modes of operation. The system enables such high power dissipation levels to be achieved utilizing a portable, battery operated arrangement.

### BRIEF DESCRIPTION OF THE DRAWINGS

FIG. 1 shows a schematic diagram of an irradiation and electrotherapy system of the present invention.

FIG. 2 shows an elevational-wiew of a wand with a laser resonator and a metal sheath used in the system of FIG. 1.

FIG. 2A shows an enlarged, elevational view of the laser resonator in the wand of FIG. 2.

FIG. 2B shows an enlarged, end view of the laser resonator in FIG. 2B.

FIG. 3 shows an elevational view of a wand with a laser resonator in an alternative embodiment to that of the wand of FIG. 2.

FIG. 3A shows an enlarged, elevational view of the laser resonator used in the wand of FIG. 3.

FIG. 3B shows an enlarged, end view of the laser resonator used in the wand of FIG. 3A.

### DETAILED DESCRIPTION OF THE PREFERRED EMBODIMENT

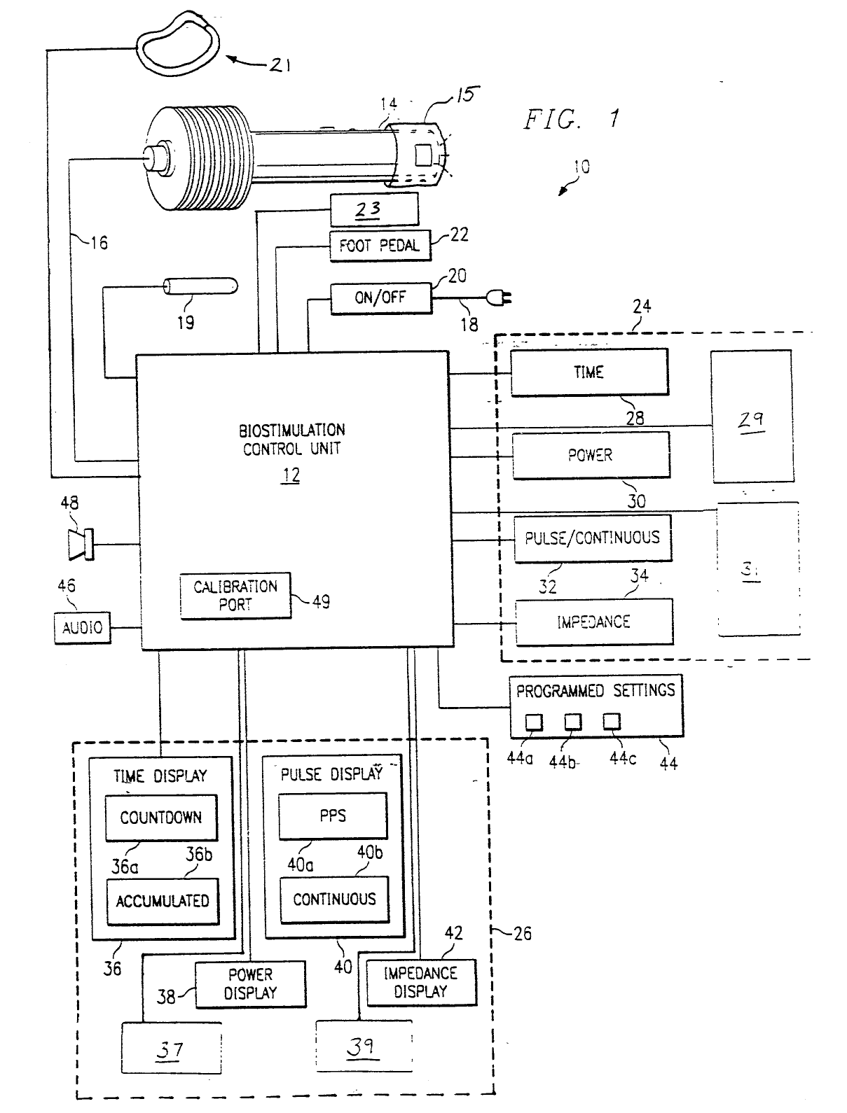

Referring to FIG. 1, the reference numeral 10 refers generally to an irradiation and electrotherapy system of the present invention which includes a biostimulation control unit 12 for controlling the operation of a hand-operated probe, i.e., a treatment wand 14, electrically connected to the control unit via a coaxial cable 16. As will be described in detail below, the wand 14 houses a laser diode capable of emitting low level reactive light for use in tissue irradiation therapy. Furthermore, the wand 14 has a metal sheath 15 located at an end of the wand 14 for use in electrotherapy involving electrical stimulation of the tissue.

The control unit 12 receives power through a power supply line 18 adapted for connection to a conventional 120-volt power outlet. A ground piece 19 is connected to the control unit 12 and is held by a patient receiving the tissue irradiation therapy to provide an electrical ground for safety purposes. Likewise, a wrist strap 21 is connected to the control unit 12 and strapped onto an arm or a leg of the patient receiving electrical stimulation. An on/off switch 20 is connected in series with the line 18 for controlling the flow of power through the line 18. A foot pedal 22 is connected to the control unit 12 and is depressible for activating the generation and emission of light from the wand 14. Likewise, a foot pedal 23 is connected to the control unit 12 and is depressible for activating the electrical current supplied to the metal sheath 15. Alternatively, the foot pedal 22 and the foot pedal 23 could be combined into one foot pedal (not shown). Activation of the laser light or the electrical current may alternatively, or additionally, be provided using a switch on the wand 14.

The control unit 12 includes setting controls 24 and corresponding setting displays 26. The setting controls 24 are utilized to select operational parameters of the control unit 12 to effect the rate of absorption and conversion to heat of tissue irradiated by the wand 14, according to desired treatment protocols as well as the amount of electrical current for electrical stimulation from the metal sheath 15. Generally, the treatment protocols provide for a rate of absorption and conversion to heat in the irradiated tissue in a range between a minimum rate sufficient to elevate the average temperature of the irradiated tissue to a level above the basal body temperature of the subject and a maximum rate which is less than the rate at which the irradiated tissue is converted to a collagenous substance. Absorption and conversion rates are enhanced by using electrotherapy simultaneously with irradiation. Alternatively, electrotherapy can be used prior to or after irradiation to enhance the absorption and conversion rates during irradiation. The treatment protocols vary time, power, electrical current and pulse/continuous mode parameters in order to achieve the desired therapeutic effects.

The setting controls 24 include a treatment time control 28, an electrical current level control 29,a power control 30, a frequency control 31 and a pulse/continuous mode control 32. Adjustments in treatment time, electrical current, power, frequency, and pulse/continuous mode operation of the wand 14 and the metal sheath 15 utilizing the controls 28-32 make possible improved therapeutic effects based upon the aforementioned treatment protocols involving one or more of these parameters. Also, an impedance control 34 is provided for adjusting an impedance measurement of the tissue to a baseline value, according to skin resistance, as discussed further below, whereby improvements in tissue condition may be monitored. It is understood that, according to the specific embodiment of the control unit 12, the setting controls 24 may include any combination of one or more of the controls 28-34.

The setting displays 26 include a time display 36, an electrical current level display 37, a power display 38, a frequency display 39, a pulse display 40 and an impedance display 42. In one embodiment, each of the displays 26 are light emitting diode (LED) displays such that the corresponding setting controls 24 can be operated to increment or decrement the settings, which are then indicated on the displays. A programmed settings control 44 is used to save setting selections and then automatically recall them for convenience, using one or more buttons 44a-44c, for example.

The time control 28 adjusts the time that laser light is emitted from the wand 14 from 0.01 to 9.99 minutes in 0.01 minute intervals, as indicated on the time display 36. The time display 36 includes a countdown display 36a and an accumulated display 36b. Once the time control 28 is set, the countdown display 36a indicates the setting so that as the wand 14 is operated the time is decremented to zero. The accumulated time display 36b increments from zero (or any other reset value) as the wand 14 is operated so that the total treatment time is displayed. The time display 36 takes into account the pulsed or continuous mode operation of the system 10.

The electrical current level control 29 varies the electrical current level supplied to the metal sheath 15 in the range of 150 microamps to 500 microamps at an isolation frequency in the range of 5 Hz to 300 Hz. The current level control 29 can also vary the electrical current supplied to the metal sheath 15 so that the electrical current is supplied in pulses corresponding to the pulse mode of operation of the irradiation treatment. The pulse has a predetermined time duration. Alternatively, the current level control 29 can vary the electrical current level so that the electrical current supplied to the metal sheath 15 varies from a high to low value within the electrical current range, during a treatment session.

The power control 30 adjusts the power dissipation level of the light from the wand 14 in a range from zero to 1000 mW, with typical operation ranging from about 10 mW to 1000 mW. The pulse/continuous mode control 32 sets the system 10 to generate light energy from the wand 14 either continuously or as a series of pulses. The control 32 may include, for example, a pulse duration rheostat (not shown) for adjusting the pulse-on or pulse-off time of the wand 14. In one implementation, the pulses-per-second (PPS) is set in a range from zero to 9995, adjustable in 5 step increments. The PPS setting is displayed on a PPS display 40a. The pulse duration may alternatively, or additionally, be displayed indicating the duty cycle of pulses ranging from 5 to 99 (e.g., 5 meaning that the laser is “on” 5% of the time).

A continuous mode display 40b is activated when the system 10 is being operated in the continuous wattage (CW) mode of operation. When the system 10 operates in CW, the energy density produced is in the range of 0.01 W/mm² to 0.04 W/mm².

An audio volume control 46 is provided for generating an audible warning tone from a speaker 48 when laser light or electrical power is being generated. Thus, for example, the tone may be pulsed when the system is operating in the pulse mode of operation.

The impedance control 34 is a sensitivity setting that is calibrated and set, according to the tissue skin resistance, to a baseline value which is then indicated on the impedance display 42. As therapy progresses the impedance readout on the display 42 changes (i.e., it decreases) thereby indicating progress of treatment.

A calibration port 49 is utilized to verify laser performance by placing the wand 14 in front of the port and operating the system 10. The port 49 determines whether the system 10 is operating within calibration specifications and automatically adjusts the system parameters.

While not shown, the control unit 12 includes digital and analog electronic circuitry for implementing the foregoing features. The details of the electronic circuitry necessary to implement these features will be readily understood by one of ordinary skill in the art in conjunction with the present disclosure and therefore will not be described in further detail.

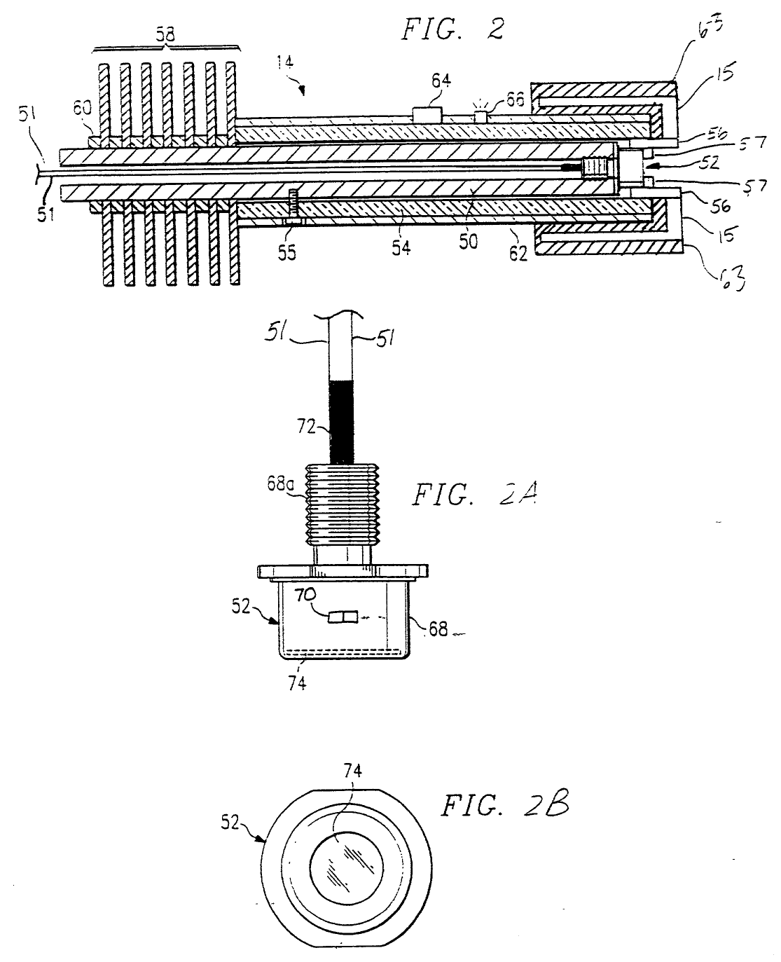

Referring to FIG. 2, the wand 14, sized to be easily manipulated by the user, includes a heat-conductive, metal bar 50 and the metal sheath 15. The bar 50 is hollow along its central axis and is threaded on its interior at a first end for receiving a fiber optic housing 52. Fiber optic cabling 51 extends from the housing 52 through the hollow axis of the bar 50 to the cable 16, FIG. 1. In the preferred embodiment, the bar 50 is copper or steel and, thus, conducts electricity for providing a ground connection for the housing 52 to the cable 16.

A glass noryl sleeve 54 is placed over the bar 50 for purposes of electrical and thermal insulation. A screw 55 extending through the sleeve 54 anchors the sleeve to the bar 50. As shown, the resonator 52 is recessed slightly within the sleeve 54. An impedance o-ring 56, formed of a conductive metal, is press-fitted into the end of the sleeve 54 so that when the wand 14 makes contact with tissue, the ring 56 and the metal sheath 15 touch the tissue. The ring 56 is electrically connected through the wand 14 to the unit 12. The ring 56 measures impedance by measuring angular DC resistance with an insulator ohmmeter, for example, of the tissue being irradiated by the wand 14, which is then displayed as impedance on the display 42. Any other suitable impedance measurement circuit may be utilized, as will be apparent to one skilled in the art. Measurements of impedance are useful in therapy to determine whether healing has occurred. For example, a baseline measurement of impedance provides an objective value of comparison wherein as the tissue heals, a lower impedance approaching the baseline is observed. The impedance value read can also be used to determine the amount of milliwattage and time of treatment appropriate for the patient.

A feedback sensor 57 is located in the end of the sleeve 54 for measuring the output from the housing 52. While not shown, the sensor 57 is connected electronically to the control unit 12 and to a feedback circuit within the control unit 12. A small percentage of the light emitted from the housing 52 is thus detected by the sensor 57 and channeled into the feedback circuit of the control unit 12 to measure and control performance. Out-of-specification temperature, electrical current, power, pulse frequency or duration is thus corrected or the system 10 is automatically turned off.

Multiple metallic fins 58 are placed over the end of the bar 50 and are separated and held in place by spacers 60 press-fitted over the bar 50. The fins 58 act as a heat sink to absorb heat from the bar 50 and dissipate it into the surrounding air. The spacers 60 placed between each fin 58 enable air to flow between the fins 58, thereby providing for increased heat transfer from the wand 14.

A casing 62 fits over the sleeve 54 and serves as a hand grip. The metal sheath 15 is coated by an insulation material 63. The insulation material 63 covers the metal sheath 15 such that a surface area of the metal sheath 15 is left exposed for contact with the tissue. The metal sheath 15 is wired in a suitable manner to the control unit 12 to receive electrical current. The insulation material also provides a friction surface to hold the metal sheath 15 onto the casing 62. Alternatively, a screw (not shown) can be placed through the metal sheath 15 and insulated therefrom to secure the metal sheath 15 in place. The casing 62 supports a switch 64 and light 66. The switch 64 is used to actuate the wand 14 by the operator, wherein the switch must be depressed for the wand 14 to operate. The switch 64 is wired in a suitable manner to the control unit 12 and is used either alone or in conjunction with the foot pedals 22 and 23. The light 66 is illuminated when the wand 14 is in operation.

As shown in FIG. 2A, the housing 52 includes a casing 68 having threads 68a configured for matingly engaging the threaded portion of the tube 50 in its first end. An Indium-doped Gallium Arsenide (In:GaAs) semiconductor diode 70 is centrally positioned in the housing 68 facing in a direction outwardly from the housing 68, and is electrically connected for receiving electric current through the threads 68a and an electrode 72 connected to the wiring 51 that extends longitudinally through the hollow interior of the bar 50, FIG. 2. The amount of Indium with which the Gallium Arsenide is doped in the diode 70 is an amount appropriate so that the diode 70, when electrically activated, generates, in the direction outwardly from the housing 68, low level reactive laser light having, at a power output level of 10-1000 mW, a fundamental wavelength ranging from, depending upon the implementation, about 630±20 nm to 1064±20 nm. Other types of diode semiconductor lasers may also be used to produce the foregoing wavelengths, e.g.,helium neon, gallium arsenide, neodymium yttrium-aluminum-garnet or the like. When activated, light is emitted in the outward direction toward a lens 74.

As shown in FIGS. 2A and 2B, the lens 74 is positioned at one end of the housing 68 in the path of the light for focusing the light onto tissue treatment areas of, for example, .5 mm² to 2 mm², and to produce in the treatment areas an energy density in the range from about 0.01 to 0.15 joules/mm². The lens 74 may be adjusted to determine depth and area of absorption.

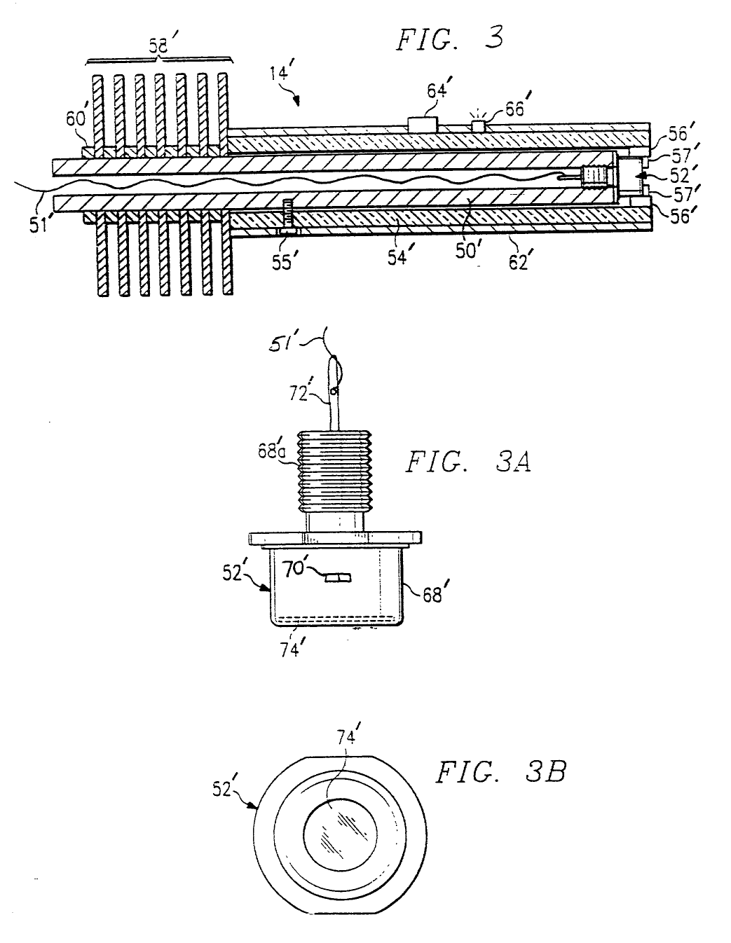

Referring to FIG. 3, in an alternative embodiment, the wand 14 is replaced by a wand 14′, sized to be easily manipulated by the user. The wand 14′ includes a heat-conductive, metal bar 50′. The bar 50′ is hollow along its central axis and is threaded on its interior at a first end for receiving a laser resonator 52′, described further below with reference to FIGS. 3A and 3B. Wiring 51′ extends from the resonator 52′ through the hollow axis of the bar 50′ for connection to the coaxial cable 16, FIG. 1. Preferably, the bar 50′ is copper or steel and thus conducts electricity for providing a ground connection for the resonator 52′ to the cable 16.

A glass noryl sleeve 54′ is placed over the bar 50′ for purposes of electrical and thermal insulation. A screw 55′ extending through the sleeve 54′ anchors the sleeve to the bar 50′. As shown, the resonator 52′ is recessed slightly within the sleeve 54′. An impedance o-ring 56′, formed of a conductive metal, is press-fitted into the end of the sleeve 54′ so that when the wand 14′ makes contact with tissue, the ring 56′ touches the tissue. The ring 56′ is electrically connected through the wand 14′ to the unit 12. The ring 56′ measures impedance by measuring angular DC resistance with an insulator ohmmeter, for example, of the tissue being irradiated by the wand 14′ which is then displayed as impedance on the display 42. Any other suitable impedance measurement circuit may be utilized, as will be apparent to one skilled in the art. Measurements of impedance are useful in therapy to determine whether healing has occurred. For example, a baseline measurement of impedance provides an objective value of comparison wherein as the tissue heals, a lower impedance approaching the baseline is observed. The impedance value read can also be used to determine the amount of milliwattage and time of treatment appropriate for the patient.

A feedback sensor 57′ is located in the end of the sleeve 54′ for measuring the output from the resonator 52′. While not shown, the sensor 57′ is connected electronically to the control unit 12 and to a feedback circuit within the control unit 12. A small percentage of the light from the resonator 52′ is thus detected by the sensor 57′ and channeled into the feedback circuit of the control unit 12 to measure and control performance of the resonator. Out-of-specification temperature, power, pulse frequency or duration is thus corrected or the system 10 is automatically turned off.

Multiple metallic fins 58′ are placed over the end of the bar 50′ and are separated and held in place by spacers 60′ press-fitted over the bar 50′. The fins 58′ act as a heat sink to absorb heat from the laser through the bar 50′ and dissipate it into the surrounding air. The spacers 60′ placed between each fin 58′ enable air to flow between the fins, thereby providing for increased heat transfer from the wand 14′.

A casing 62′ fits over the sleeve 54′ and serves as a hand grip. The casing 62′ supports a switch 64′ and light 66′. The switch 64′ is used to actuate the wand 14′ by the operator wherein the switch must be depressed for the wand 14′ to operate. The switch 64′ is wired in a suitable manner to the control unit 12 and is used either alone or in conjunction with the foot pedal 22. The light 66′ is illuminated when the wand 14′ is in operation.

As shown in FIG. 3A, the laser resonator 52′ includes a housing 68′ having threads 68′a configured for matingly engaging the threaded portion of the bar 50′ in its first end. An Indium-doped Gallium Arsenide (In:GaAs) semiconductor diode 70′ is centrally positioned in the housing 68 facing in a direction outwardly from the housing 68′, and is electrically connected for receiving electric current through the threads 68′a and an electrode 72′ connected to the wiring 51′ that extends longitudinally through the hollow interior of the bar 50′, FIG. 3. The amount of Indium with which the Gallium Arsenide is doped in the diode 70′ is an amount appropriate so that the diode 70′, when electrically activated, generates, in the direction outwardly from the housing 68′, low level reactive laser light having, at a power output level of 100-1000 mW, a fundamental wavelength ranging from, depending upon the implementation, about 1064±20 nm to 2500±20 nm in the near-infrared region of the electromagnetic spectrum. Other types of diode semiconductor lasers may also be used to produce the foregoing wavelengths, e.g., Helium Neon, GaAs or the like.

As shown in FIGS. 3A and 3B, a lens 74′ is positioned at one end of the housing 68 in the path of the generated laser light for focusing the light onto tissue treatment areas of, for example, 0.5 mm² to 2 mm², and to produce in the treatment areas an energy density in the range from about 0.01 to about 0.15 joules/mm². The lens 74′ may be adjusted to determine depth and area of absorption.

The operating characteristics of the diode 70′ are an output power level of 100-100 mW, a center fundamental wavelength of 1064±20 nm to 2500±20 nm, with a spectral width of about 5 nm, a forward current of about 1500 milliamps, and a forward voltage of about 5 volts at the maximum current.

In operation, the switch 20 is closed (i.e., turned on) to power up the control unit 12, at which time the displays become illuminated, thereby indicating that the control unit is receiving power. The time control 28 is set for specifying a desired duration of time for laser treatment, which time is displayed on the countdown display 36a. The mode control 34 is set for specifying whether the light from the laser diode 70, FIG. 2, or the laser diode 70′, FIG. 3A, is to be generated in the continuous or the pulsed mode. If the pulsed mode is selected, then the duration of the pulse on-time/off time is specified and the pulses-per-second (and the pulse duty cycle if appropriate) is displayed on the PPS display 40a. If the continuous mode instead is chosen, the continuous mode display 40b is illuminated. It can be appreciated that the mode and the pulse time-on and time-off settings affect the intensity of the treatment provided. Likewise, the current settings can be in a continuous mode or a pulse mode corresponding to the mode selected for the irradiation treatment. The amount of power is further set by the power control 30, and displayed on the power display 38. It can be appreciated that the power, duration and pulse intensity of treatment is thus selectable by the unit 12 and is to be determined by treatment protocols relating to the character of the tissue to be treated, the depth of penetration desired, the acuteness of the injury, and the condition of the patient. The audio volume control 46 can be adjusted to control the volume of the tone generated from the speaker 48. The tissue impedance display 42 indicates an impedance value for tissue in contact with it and can be calibrated to a baseline set for the patient by applying the wand 14 or 14′ to surrounding non-damaged tissue and then when the wand 14 or 14′ is applied to the damaged tissue, an impedance value (much higher than the baseline) will be indicated and hopefully reduced over time, through treatment, to the baseline value.

After the time, electrical current and isolation frequency, power, and/or mode (continuous wattage or pulsed at a selected intensity) selections are made, the wands 14 and 14′ may be directed into the calibration port 49 to verify the accuracy of the system. The wand 14 or 14′ may then be applied to patient tissue for therapy, the foot pedals 22 and 23 and/or the switch 64 may be depressed to cause therapeutic light energy and/or electrical stimulation to be generated from the wands 14 or 14′. As an indication that electrical current or laser light energy is being generated, an audible tone is generated from the speaker 32. In accordance with the foregoing specification for the laser diode 70, the light is generated at a fundamental wavelength of 630 nm to 1064 nm at a power output level from about 10-100 mW. The electrotherapy operates in the range of 150 microamps to 500 microamps at a frequency in the range of 5 Hz to 18 Hz. In accordance with the foregoing specification of the laser diode 70′, the laser light energy is generated at a fundamental wavelength of 1064 nm at an output power level from about 100-800 mW. In other implementations the laser light wavelength may be as high as about 2500 nm and the power up to 1000 mW.

The generated energies are applied to regions of the body where decreased muscle spasms, increased circulation, decreased pain, or enhanced tissue healing is desired. The surface of the tissue in the region to be treated is demarcated to define an array of grid treatment points, each of which points identifies the location of an aforementioned small treatment area. Each small treatment area is irradiated with the light and/or electrically stimulated to produce the desired therapeutic effect. Because the light is coherent, a variable energy density of the light of about 0.01 to 0.15 joules/mm² is obtained as the light passes through the lens 74 or 74′ and converges onto each of the small treatment areas. Energy of the irradiation is controlled by the power control 30 and applied (for durations such as 1 minute to 3 minutes, continuous wattage or pulsed, for example) as determined by treatment protocols, to cause the amount of optical energy absorbed and converted into heat to be within a range bounded by a minimum absorption rate sufficient to elevate the average temperature of the irradiated tissue to a level which is above the basal body temperature, but which is less than the absorption rate at which tissue is converted into a collagenous substance. The beam wavelength, spot or beam size, electrical current and isolation frequency, power dissipation level, and time exposure are thus carefully controlled to produce in the irradiated and/or electrically stimulated tissue a noticeable warming effect which is also limited to avoid damaging the tissue.

The present invention has several advantages. For example, by using electrical stimulation with irradiation caused by short wavelength light, a deeper penetration can be achieved that was not previously possible. Additionally, by using an In:GaAs diode laser to generate the laser beam energy, the laser source can be made sufficiently small to fit within the hand-held wands 14 or 14′, thereby obviating the need for a larger, more expensive laser source and the fiber optic cable necessary to carry the laser energy to the treatment tissue. The In:GaAs diode laser can also produce greater laser energy at a higher power dissipation level than lasers of comparable size. Furthermore, construction of the wands 14 and 14′ including the fins 58 and 58′, respectively, provides for the dissipation from the wands 14 and 14′ heat generated during operation.

A further advantage is that therapeutic treatment by the foregoing has been shown to reduce pain in soft tissue, reduce inflammation, and enhance healing of damaged tissue by the stimulation of microcirculation, without subjecting the living tissue to damaging thermal effects. This phenomenon is due to certain physiological mechanisms in the tissue and at the cellular level that occur when the above process is used. In the evaluation of the microcirculatory system, for example, it has been demonstrated that the blood vessel walls possess photosensitivity. When the blood vessel walls are exposed to laser irradiation as set forth above, the tonus is inhibited in smooth myocytes, thus increasing the blood flow in the capillaries. Other effects which have been observed are: peripheral capillarid neovascularization, reduction of blood platelet aggregation, reduction of O₂ from the triplet to the singlet form which allows for greater oxygenation of the tissue, reduction of buffer substance concentration in the blood, stabilization of the indices of erythrocyte deformation, reduction of products of perioxidized lipid oxygenation of the blood. Other effects which have been observed are increased index of antithrombin activity, stimulation of the enzymes of the antioxidant system such as superoxide dismutase and catalase. An increase in the venous and lymph and outflow from the irradiated region has been observed. The tissue permeability in the area is substantially enhanced. This assists in the immediate reduction of edema and hematoma concentrations in the tissue. At the cellular level, the mitochondria have also been noted to produce increased amounts of ADP with subsequent increase in ATP. There also appears to be an increased stimulation of the calcium and sodium pumps at the tissue membrane at the cellular level.

At the neuronal level, the following effects have been observed as a result of the foregoing therapeutic treatment. First, there is an increased action potential of crushed and intact nerves. The blood supply and the number of axons is increased in the irradiated area. Inhibition of scar tissue is noticed when tissue is lazed. There is an immediate increase in the membrane permeability of the nerve. Long term changes in the permeability of calcium and potassium ions through the nerve for at least 120 days have been observed. The RNA and subsequent DNA production is enhanced. Singlet O₂ is produced which is an important factor in cell regeneration. Pathological degeneration with nerve injury is changed to regeneration. Both astrocytes and oligodedrocytes are stimulated which causes an increased production of peripheral nerve axons and myelin.

Phagocytosis of the blood cells is increased, thereby substantially reducing infection. There also appears to be a significant anti-inflammatoryphenomena which provides a decrease in the inflammation of tendons, nerves, bursae in the joints, while at the same time yielding a strengthening of collagen. There is also an effect on the significant increase of granulation tissue in the closure of open wounds under limited circulation conditions.

Analgesia of the tissue has been observed in connection with a complex series of actions at the tissue level. At the local level, there is a reduction of inflammation, causing a reabsorption of exudates. Enkephalins and endorphins are recruited to modulate the pain production both at the spinal cord level and in the brain. The serotnogenic pathway is also recruited. While it is not completely understood, it is believed that the irradiation of the tissue causes the return of an energy balance at the cellular level which is the reason for the reduction of pain.

It is understood that several variations may be made in the foregoing without departing from the scope of the invention. For example, any number of fins 58 or 58′ may be utilized as long they dissipate sufficient heat from the wand 14 or 14′, respectively, so that the user may manipulate the wand without getting burned. The setting controls 24 may be used individually or in combination and the information displayed on the displays 26 may vary. Other diode laser structures may be utilized to produce the desired effects.

Although illustrative embodiments of the invention have been shown and described, a wide range of modification, change, and substitution is contemplated in the foregoing disclosure and in some instances, some features of the present invention may be employed without a corresponding use of the other features. Accordingly, it is appropriate that the appended claims be construed broadly and in a manner consistent with the scope of the invention.

What is claimed is:

1. A system for treating biological tissue of a subject without exposing the tissue to damaging effects, the system comprising:
a manipulable wand for contact with the tissue;
a diode laser for emitting irradiation light disposed in the wand for irradiating the tissue with coherent optical energy having a wavelength less than one thousand and sixty four nanometers; and
setting controls coupled to the wand for operating the irradiation emitted from the laser diode to achieve a rate of absorption and conversion to heat in the irradiated tissue in a range between a minimum rate sufficient to elevate the average temperature of the irradiated tissue to a level above the basal body temperature of the subject, and a maximum rate which is less than the rate at which the irradiated tissue is converted into a collagenous substance.

2. The system of claim 1 further comprising:
a metal sheath coupled to the setting controls and mounted onto an end of the wand for emitting electrical current to stimulate the tissue; and
a strap coupled to the setting controls for providing a ground for the metal sheath, the strap being tied to the wrist of a patient while the patient is receiving electrical stimulation.

3. The system of claim 2 wherein the irradiation and the electrical stimulation occur simultaneously.

4. The system of claim 2 wherein the setting controls comprise a time control for setting the irradiation treatment time, a current level control for setting the level of electrical stimulation, and a power control for setting the power level of the emitted light.

5. The system of claim 2 wherein the setting controls comprise a pulse/continuous mode control for setting the diode laser to operate in a continuous wattage mode of operation or in a pulsed wattage mode of operation.

6. The system of claim 5 wherein in the continous mode, the energy density produced is in the range of 0.01 W/mm² to 0.04 W/mm².

7. The system of claim 5 wherein in the pulsed wattage mode, the pulse/continuous mode control selects the number of light pulses-per-second emitted from the laser diode.

8. The system of claim 5 wherein in the pulsed wattage mode, the pulse/continuous mode control selects the ratio of on-to-off pulsing.

9. The system of claim 5 wherein the setting controls include an impedance control for calibrating an impedance reading of the tissue.

10. The system of claim 5 wherein the setting controls include a programmed setting control for saving and recalling selected settings.

11. The system of claim 2 further comprising means for focussing the energy emitted by the diode laser to a treatment area in the range of about 0.5 m² to about 2 mm².

12. The system of claim 2 wherein the wand comprises a conductive bar supporting the diode laser at one end thereof, an insulative sleeve over the bar, and cooling fins connected to the bar for transferring heat to the surrounding air.

13. The system of claim 2 wherein the wand includes an impedance sensor for contact with the tissue for measuring impedance of the tissue being treated.

14. The system of claim 2 wherein the wand includes a feedback sensor for measuring the output from the diode laser, the sensor being connected to a feedback circuit for monitoring the accuracy of the setting controls.

15. The apparatus of claim 2 further comprising a time display for displaying the treatment time remaining for a treatment time selected using the setting controls.

16. The apparatus of claim 2 further comprising a power display or displaying a treatment power output selected using the setting controls.

17. The system of claim 2 further comprising a calibration port for calibrating the settings of the wand by placing the wand in proximity to the port.

18. A method for treating biological tissue of a subject using an irradiation and electrotherapy system, the method comprising:
manipulating a wand in contact with the tissue, the wand including a laser diode disposed in the wand for irradiating the tissue with coherent optical energy having a wavelength less than one thousand and sixty four nanometers and a metal sheath disposed about the contact end of the wand for electrically stimulating the tissue; and
operating the wand, using setting controls of the system, to achieve a rate of absorption and conversion to heat in the irradiated tissue in a range between a minimum rate sufficient to elevate the average temperature of the irradiated tissue to a level above the basal body temperature of the subject, and a maximum rate which is less than the rate at which the irradiated tissue is converted into a collagenous substance.

19. The method of claim 18 wherein the step of operating the wand using the setting controls comprises setting the irradiation treatment time, the level of electrical current, the power level, isolation frequency of the electrical current, and the pulse/continuous operating mode of the wand to selected parameters according to a treatment protocol.

20. The method of claim 18 wherein the coherent optical energy emitted from the wand occurs simultaneous to the electrical stimulation.

21. The method of claim 18 wherein the electrical current emitted from the wand is less than about five hundred microamps and in the range of about five Hertz to about eighteen Hertz.

22. The method of claim 18 further comprising focussing the energy emitted from the wand to a treatment area in the range of about 0.5 mm²to about 2 mm².

23. A diode laser irradiation system for treating biological tissue of a subject without exposing the tissue to damaging thermal effects, the system comprising:
a manipulable wand for contact with the tissue;
a diode laser disposed in the wand for irradiating the tissue with coherent optical energy at a power output level of less than one thousand milliwatts; and
laser setting controls for operating the diode laser to achieve a rate of absorption and conversion to heat in the irradiated tissue in a range between a minimum rate sufficient to elevate the average temperature of the irradiated tissue to a level above the basal body temperature of the subject, and a maximum rate which is less than the rate at which the irradiated tissue is converted into a collagenous substance.

24. The system of claim 23 wherein the laser setting controls comprise a time control for setting the irradiation treatment time and a power control for setting the power revel of the diode laser.

25. The system of claim 23 wherein the laser setting controls comprise a pulse/continuous mode control for setting the diode laser to operate in a continuous wattage mode of operation or in a pulsed wattage mode of operation.

26. The system of claim 25 wherein in the continous mode, the energy density produced is in the range of 0.01 W/mm² to 0.04 W/mm².

27. The system of claim 25 wherein in the pulsed wattage mode, the pulse/continuous mode control selects the number of light pulses-per-second emitted by the laser diode.

28. The system of claim 25 wherein in the pulsed wattage mode, the pulse/continuous mode control selects the ratio of on-to-off pulsing of the laser diode.

29. The system of claim 23 wherein the laser setting controls include an impedance control for calibrating an impedance reading of the tissue.

30. The system of claim 23 wherein the laser setting controls include a programmed setting control for saving and recalling s elected laser settings.

31. The system of claim 23 wherein the coherent optical energy emitted by the diode laser has a wavelength of less than about 2500 nanometers.

32. The system of claim 23 wherein the coherent optical energy emitted by the diode laser has a wavelength of about 1064 nanometers.

33. The system of claim 23 further comprising means for focussing the energy emitted by the diode laser to a treatment area in the range of about 0.5 mm² to about 2 mm².

34. The system of claim 23 wherein the diode laser is an Indium doped Gallium Arsenide diode laser.

35. The system of claim 23 wherein the wand comprises a conductive bar supporting the diode laser at one end thereof, an insulative sleeve over the bar, and cooling fins connected to the bar for transferring heat generated by the diode laser to the surrounding air.

36. The system of claim 23 wherein the wand includes an impedance sensor for contact with the tissue for measuring impedance of the tissue being treated.

37. The system of claim 23 wherein the wand includes a feedback sensor for measuring the output of the diode laser, the sensor being connected to a feedback circuit for monitoring the accuracy of the setting controls.

38. The apparatus of claim 23 further comprising a time display for displaying the treatment time remaining for a treatment time selected using the setting controls.

39. The apparatus of claim 23 further comprising a power display or displaying a treatment power output selected using the setting controls.

40. The system of claim 23 further comprising a calibration port for calibrating the settings of the diode laser by placing the wand in proximity to the port.

41. A method for treating biological tissue of a subject using a diode laser irradiation system, the method comprising:
manipulating a wand in contact with the tissue, the wand including a diode laser disposed in the wand for irradiating the tissue with coherent optical energy at a power output level of less than one thousand milliwatts; and
operating the diode laser, using laser setting controls of the system, to achieve a rate of absorption and conversion to heat in the irradiated tissue in a range between a minimum rate sufficient to elevate the average temperature of the irradiated tissue to a level above the basal body temperature of the subject, and a maximum rate which is less than the rate at which the irradiated tissue is converted into a collagenous substance.

42. The method of claim 41 wherein the step of operating the diode laser using the laser setting controls comprises setting the irradiation treatment time, the power level and the pulse/continuous operating mode of the diode laser to selected parameters according to a treatment protocol.

43. The method of claim 41 wherein the coherent optical energy emitted by the diode laser has a wavelength of less than about 2500 nanometers.

44. The method of claim 41 wherein the coherent optical energy emitted by the diode laser has a wavelength of about 1064 nanometers.

45. The method of claim 41 further comprising focussing the energy emitted by the diode laser to a treatment area in the range of about 0.5 mm² to about 2 mm².
